(19) United States
(12) Patent Application Publication
(10) Pub. No.: US 2005/0033102 A1
(43) Pub. Date: Feb. 10, 2005

(54) Supported ionic liquid and the use thereof in the disproportionation of isopentane

(76) Inventors: Bruce B. Randolph, Bartlesville, OK (US); Edward Lawrence Sughrue, Bartlesville, OK (US); Glenn W. Dodwell, Bartlesville, OK (US)

Correspondence Address: RICHMOND, HITCHCOCK, FISH & DOLLAR, P.O. Box 2443, Bartlesville, OK, 74005, US

(21) Appl. No.: 10/635,759

(22) Filed: Aug. 6, 2003

(51) Int. Cl. C07C 6/08

(52) U.S. Cl. 585/708

(57) ABSTRACT

A catalyst system containing an ionic liquid dispersed on a support having an average pore diameter greater than about 225 Å is disclosed. The catalyst system is employed in a process to disproportionate a C₅ paraffin.

### BACKGROUND OF THE INVENTION

The invention relates to catalyst systems useful in hydrocarbon upgrading processes and to methods for their use. In another aspect, this invention relates to processes for the disproportionation of a C₅ paraffin.

It is known that ionic liquids can be used in various hydrocarbon conversion processes. However, we have found an ionic liquid dispersed on a support having an average pore diameter greater than about 225 angstroms is unexpectedly effective at disproportionating isopentane.

### SUMMARY OF THE INVENTION

It is an object of this invention to provide an improved supported ionic liquid which when used in the disproportionation of a C₅ paraffin results in increased product formation.

Another object of this invention is to provide an improved process for the disproportionation of a C₅ paraffin in which the yield of disproportionation products is increased.

The inventive catalyst system comprises an ionic liquid dispersed on a support having an average pore diameter greater than about 225 angstroms. The inventive catalyst system can be used in the disproportionation of a C₅ paraffin by contacting a C₅ paraffin, under conversion conditions, with the inventive catalyst system.

Other objects and advantages of the invention will become apparent from the detailed description and the appended claims.

### DETAILED DESCRIPTION OF THE INVENTION

The hydrocarbon feed stream of the process of this invention can be any hydrocarbon-containing mixture that comprises at least one C₅ paraffin such as n-pentane, 2-methylbutane, neopentane, or mixtures thereof, and an initiator selected from the group consisting of an olefin, alkyl halides, and combinations thereof. The olefin or alkyl halide preferably has in the range of from 2 to 20 carbon atoms per molecule, more preferably has in the range of from 3 to 6 carbon atoms per molecule, and most preferably has in the range of from 4 to 5 carbon atoms per molecule. Generally, the feed contains more than about 50 weight-percent C₅ paraffin, preferably about 60-100 weight percent C₅ paraffin, and more preferably about 75-90 weight percent C₅ paraffin. The feed can contain other hydrocarbons that do not interfere with the process of this invention, i.e. minor amounts of other alkanes, such as n-butane, isobutane, n-hexane and the like, and alkenes (monoolefins).

The inventive catalyst system comprises, consists of, or consists essentially of an ionic liquid dispersed on a support having an average pore diameter greater than about 225 angstroms, preferably greater than about 250 angstroms, and more preferably greater than about 275 angstroms. The support preferably has a surface area less than about 700 m² per gram and is preferably non-crystalline. The support is most preferably a silica.

The ionic liquid comprises, consists of, or consists essentially of a cation and an anion. The cation is preferably selected from the group consisting of ions defined by the formulas: 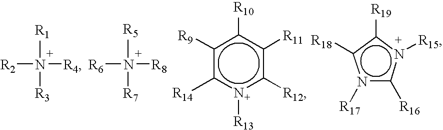
and combinations of any two or more thereof, wherein:
R₁, R₂, R₃, R₅, R₆ and R₇ are selected from saturated and unsaturated hydrocarbons containing from 1 to 7 carbon atoms per molecule; R₄, R₈, R₉, R₁₀, R₁₁, R₁₂, R₁₃, R₁₄, R₁₅, R₁₆, R₁₇, R₁₈ and R₁₉ are selected from saturated and unsaturated hydrocarbons containing from 1 to 7 carbon atoms per molecule, and hydrogen.

The anion is selected from the group consisting of halides of: Group IIIA metals, copper, zinc, iron, phosphorus and combinations thereof. More preferably, the anion is selected from the group consisting of chlorides of aluminum, gallium, copper, zinc, and iron; fluorides of phosphorus and boron, and combinations thereof.

The ionic liquid preferably has the general formula R₁R₂R₃ NH⁺AlₙCl₃ₙ₊₁⁻, wherein n=1, 2, or 3; and more preferably has the general formula (CH₃)₃NH⁺Al₂ Cl₇⁻.

The inventive process comprises, consists of, or consists essentially of, a) contacting, under conversion conditions, the hydrocarbon feed stream with the inventive catalyst system; and b) withdrawing a product stream comprising a C₄ paraffin which is preferably isobutane and at least one C₆ paraffin which is preferably a hexane isomer.

The conversion conditions include a temperature in the range of from about 100° F. to about 1000° F., preferably in the range of from about 140° F. to about 250° F., and more preferably in the range of from about 150° F. to about 220° F.

The following examples are presented to further illustrate this invention and are not to be construed as unduly limiting its scope.

### EXAMPLE I

For Inventive Run 1, 7.38 grams of AlCl₃ were mixed with 2.71 grams of trimethylammonium chloride (N(CH₃)₃:HCl) (at approximately 2 equivalents AlCl₃ and 1 equivalent (N(CH₃)₃:HCl)) to form an ionic liquid. A 1.98 gram quantity of silica spheres, having a surface area greater than about 400 m²/g, a pore volume of 3.0 cc/g, and an average pore diameter of 308 Å, were added to the ionic liquid along with 17.01 grams of the inert support Alundum alumina to form a mixture. The mixture was then charged to a reactor.

An isopentane feed was charged to the reactor at varying reactor temperatures and liquid hourly space velocities. Results of such are presented in Table 1.

TABLE I

| TOS, Hrs | | | | | | | | | |
|---|---|---|---|---|---|---|---|---|---|
| Rx Temp, | | | | | | | | | |
| ° F. | | 1 | 2 | 3 | 4 | 5 | 6 | 7 | 8 |
| LHSV, hr-1 | Feed | 102.74 | 102.02 | 140.52 | 151.32 | 199.22 | 201.92 | 201.32 | 201.42 |
| Component | Wt % | Wt % | Wt % | Wt % | Wt % | Wt % | Wt % | Wt % | Wt % |
| C3 | 0.000 | 0.000 | 0.000 | 0.000 | 0.011 | 0.112 | 0.266 | 0.174 | 0.127 |
| iC4 | 0.057 | 2.851 | 3.494 | 4.425 | 16.507 | 32.702 | 33.686 | 28.550 | 24.162 |
| NC4 | 0.000 | 0.084 | 0.086 | 0.088 | 0.251 | 1.346 | 2.174 | 1.279 | 0.870 |
| NeoC5 | 0.198 | 0.192 | 0.192 | 0.193 | 0.195 | 0.199 | 0.198 | 0.195 | 0.194 |
| iC5 | 97.070 | 88.936 | 87.389 | 85.682 | 58.319 | 29.846 | 27.090 | 32.806 | 41.167 |
| NC5 | 0.421 | 0.616 | 0.704 | 0.905 | 2.897 | 4.884 | 5.026 | 4.194 | 3.417 |
| C5= | 2.166 | 0.016 | 0.268 | 0.016 | 0.027 | 0.020 | 0.019 | 0.037 | 0.023 |
| Unk C3-C5 | 0.087 | 0.000 | 0.010 | 0.000 | 0.002 | 0.000 | 0.001 | 0.001 | 0.013 |
| 22DMC4 | 0.000 | 0.010 | 0.016 | 0.027 | 0.606 | 2.000 | 1.860 | 0.994 | 0.619 |
| 23DMC4 | 0.000 | 0.396 | 0.495 | 0.690 | 2.142 | 2.682 | 2.629 | 3.085 | 3.060 |
| 2MC5 | 0.000 | 1.338 | 1.605 | 2.124 | 5.804 | 7.168 | 7.250 | 8.608 | 8.689 |
| 3MC5 | 0.000 | 0.625 | 0.750 | 0.994 | 2.874 | 3.658 | 3.836 | 4.587 | 4.642 |
| NC6 | 0.000 | 0.013 | 0.018 | 0.027 | 0.461 | 1.327 | 1.611 | 1.201 | 0.816 |
| Unk C6 | 0.000 | 0.001 | 0.017 | 0.000 | 0.000 | 0.016 | 0.015 | 0.025 | 0.014 |
| Total C6 | | | | | | | | | |
| Par. | 0.000 | 2.382 | 2.883 | 3.862 | 11.886 | 16.834 | 17.186 | 18.475 | 17.826 |
| C7+ | 0.000 | 4.923 | 4.956 | 4.830 | 9.905 | 14.043 | 14.339 | 14.263 | 12.187 |
| Total | 100.000 | 100.000 | 100.000 | 100.000 | 100.000 | 100.000 | 100.000 | 100.000 | 100.000 |
| Moles C4 | | 0.050 | 0.062 | 0.078 | 0.288 | 0.586 | 0.617 | 0.513 | 0.431 |
| Moles C6 | | 0.028 | 0.033 | 0.045 | 0.138 | 0.195 | 0.199 | 0.214 | 0.207 |
| iC5 Conv. | | 8.38 | 9.97 | 11.73 | 39.92 | 69.25 | 72.09 | 66.20 | 57.59 |

The data in Table I demonstrate that a catalyst system including an ionic liquid dispersed on silica spheres having an average pore diameter greater than about 225 Å (specifically, 308 Å) results in significant isopentane conversion, with even higher conversions at reactor temperatures in excess of 150° F.

For Runs 2 through 5, the catalysts were made from about 2 equivalents AlCl₃ and about 1 equivalent N(CH₃)₃:HCl to generate an ionic liquid to which silica supports were added, as shown in Table II. An isopentane feed comprising about 98.2-98.4 wt. % isopentane, about 0.8-1.4 wt. % C₅; ˜0.2 wt. % neo C₅=and about 0.13-0.15 wt. % C₄ paraffins, was charged to each reactor at an LHSV of 2 hr. Results of the conversions are presented in Table II.

TABLE II

| Silica Support | A¹ | B₁² | B₂² | B₃² |
|---|---|---|---|---|
| Surface Area, m2/g | 269 | 321 | 403 | 537 |
| Ave Pore Diam, | 164 | 372 | 308 | 228 |
| Angstroms | | | | |
| Ave Pore Vol, cc/g | 1.1 | 2.98 | 3.10 | 3.06 |
| Rx Temp, ° F. | 229.0 | 200.0 | 201.9 | 200.7 |
| IC5 Conversion, wt. % | 30.5 | 76.0 | 72.2 | 53.5 |

¹A = Davison G-57 grade
²B = Silica microspheres from Philadelphia Quartz

The data in Table II demonstrate that ionic liquid catalyst systems which have silica supports with higher average pore diameters result in higher isopentane conversion as compared to ionic liquid catalyst systems which have lower average pore diameter silica supports.

For Inventive Run 6, 2.19 grams of N(CH₃)₃:HCl were added to 7.92 grams of GaCl₃ to form an ionic liquid. A 2.05 gram quantity of silica spheres, having a surface area greater than about 400 m²/g, a pore volume of 3.0 cc/g, and an average pore diameter of 308 Å, were added to the ionic liquid along with 17.94 grams of Alundum alumina to form a mixture. The mixture was then charged to a reactor. An isopentane feed, as shown in Table III, was charged to the reactor. Results of such are presented in Table III.

TABLE III

| g catalyst | 12.16 | | | | |
|---|---|---|---|---|---|
| mL Catalyst | 12.5 | | | | |
| H2 Rate, sccm | 0 | 0 | 0 | 0 | 0 |
| Feed Rate, mL/hr | 25 | 25 | 25 | 25 | 25 |
| LHSV, hr-1 | — | 2 | 2 | 2 | 2 |
| Rx Temp, ° F. | 193.3 | 202.5 | 204.0 | 203.4 | 202.4 |
| TOS, hrs. | Feed | 2 | 3 | 4 | 5 |
| C3 | 0 | 0.050 | 0.047 | 0.035 | 0.028 |
| iC4 | 0.057 | 17.014 | 17.294 | 16.137 | 15.275 |
| nC4 | 0 | 0.274 | 0.261 | 0.227 | 0.206 |
| NeoC5 | 0.198 | 0.199 | 0.201 | 0.200 | 0.199 |
| iC5 | 97.07 | 54.220 | 54.221 | 56.733 | 58.573 |
| nC5 | 0.421 | 1.837 | 1.823 | 1.693 | 1.591 |
| C5= | 2.166 | 0 | 0 | 0 | 0 |
| UNK C₃—C₅ | 0.087 | 0.039 | 0.041 | 0.046 | 0.052 |
| 22DMC4 | — | 0.165 | 0.157 | 0.123 | 0.099 |
| 23DMC4 | — | 3.328 | 3.348 | 3.188 | 3.050 |
| 2MC5 | — | 10.622 | 10.654 | 10.369 | 10.172 |
| 3MC5 | — | 5.695 | 5.709 | 5.562 | 5.457 |
| nC6 | — | 0.320 | 0.312 | 0.262 | 0.225 |
| UNKC6 | — | 0 | 0.013 | 0.020 | 0.022 |
| Total C6 Paraffin | — | 20.170 | 20.193 | 19.524 | 19.025 |
| C7+ | — | 6.197 | 5.920 | 5.404 | 5.051 |
| TOTAL | 100.00 | 100 | 100 | 100 | 100 |
| IC₅ Conversion | — | 44.1 | 44.1 | 41.6 | 39.7 |

1. A catalyst system comprising an ionic liquid dispersed on a support having an average pore diameter greater than about 225 Å.

2. A catalyst system in accordance with claim 1 wherein said support has a surface area less than about 700 m²/gram.

3. A catalyst system in accordance with claim 1 wherein said support is non-crystalline.

4. A catalyst system in accordance with claim 1 wherein said support is non-crystalline and has a surface area less than about 700 m²/gram.

5. A catalyst system in accordance with claim 1 wherein said support is silica.

6. A catalyst system in accordance with claim 1 wherein said ionic liquid comprises a cation and an anion; wherein said cation is selected from the group consisting of ions defined by the formulas: 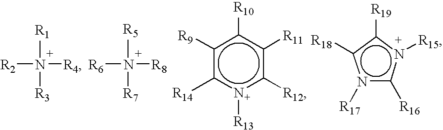
and combinations of any two or more thereof, wherein:
R₁, R₂, R₃, R₅, R₆, and R₇ are selected from saturated and unsaturated hydrocarbons containing from 1 to 7 carbon atoms per molecule;
R₄, R₈, R₉, R₁₀, R₁₁, R₁₂, R₁₃, R₁₄, R₁₅, R₁₆, R₁₇, R₁₈, and R₁₉ are selected from saturated and unsaturated hydrocarbons containing from 1 to 7 carbon atoms per molecule, and hydrogen; and
wherein said anion is selected from the group consisting of halides of: Group IIIA metals, copper, zinc, iron and phosphorus.

7. A catalyst system in accordance with claim 6 wherein said anion is selected from the group consisting of AlCl₄⁻, Al₂Cl₇⁻, Al₃Cl₁₀⁻, GaCl₄⁻, Ga₂Cl₇⁻, Ga₃Cl₁₀⁻, CuCl₂⁻, Cu₂Cl₃⁻, Cu₃Cl₄⁻, ZnCl₃⁻, FeCl₃⁻, FeCl₄⁻, Fe₃Cl₇⁻, PF₆⁻, and BF₄⁻.

8. A catalyst system in accordance with claim 6 wherein said ionic liquid has the formula R₁R₂R₃NH⁺Al₂Cl₇⁻.

9. A catalyst system in accordance with claim 6 wherein said ionic liquid has the formula (CH₃)₃NH⁺Al₂Cl₇⁻.

10. A catalyst system in accordance with claim 1 wherein a Group VIII metal compound is dispersed in said ionic liquid.

11. A catalyst system in accordance with claim 10 wherein said Group VIII metal compound comprises a platinum compound.

12. A process comprising:
a) contacting, under conversion conditions, a hydrocarbon feed stream comprising a C₅ paraffin and an initiator with a catalyst system comprising an ionic liquid dispersed on a support; and
b) withdrawing a product stream comprising a C₄ paraffin and at least one C₆ paraffin.

13. A process in accordance with claim 12 wherein said support has an average pore diameter greater than about 225 Å.

14. A process in accordance with claim 12 wherein said support has a surface area less than about 700 m²/gram.

15. A process in accordance with claim 12 wherein said support is non-crystalline.

16. A process in accordance with claim 12 wherein said support is non-crystalline, has an average pore diameter greater than about 225 Å, and has a surface area less than about 700 m²/gram.

17. A process in accordance with claim 12 wherein said support is silica.

18. A process in accordance with claim 12 wherein said ionic liquid comprises a cation and an anion; wherein said cation is selected from the group consisting of ions defined by the formulas: 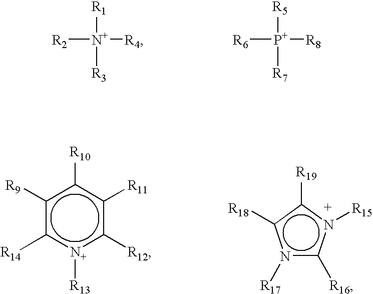
and combinations of any two or more thereof, wherein:
R₁, R₂, R₃, R₅, R₆, and R₇ are selected from saturated and unsaturated hydrocarbons containing from 1 to 7 carbon atoms per molecule;
R₄, R₅, R₉, R₁₀, R₁₁, R₁₂, R₁₃, R₁₄, R₁₅, R₁₆, R₁₇, R₁₈, and R₁₉ are selected from saturated and unsaturated hydrocarbons containing from 1 to 7 carbon atoms per molecule, and hydrogen; and
wherein said anion is selected from the group consisting of halides of: Group IIIA metals, copper, zinc, iron and phosphorus.

19. A process in accordance with claim 18 wherein said anion is selected from the groups consisting of AlCl₄⁻, Al₂C₇⁻, Al₃Cl₁₀⁻, GaCl₄⁻, Ga₂Cl₇⁻, Ga₃Cl₁₀⁻, CuCl₂⁻, Cu₂Cl₃⁻, Cu₃Cl₄⁻, ZnCl₃⁻, FeCl₃⁻, FeCl₄⁻, Fe₃Cl₇⁻, PF₆⁻, and BF₄⁻.

20. A process in accordance with claim 18 wherein said ionic liquid has the formula R₁R₂R₃NH⁺Al₂Cl₇⁻.

21. A process in accordance with claim 18 wherein said ionic liquid has the formula (CH₃)₃NH⁺Al₂Cl₇⁻.

22. A process in accordance with claim 12 wherein said hydrocarbon feed stream comprises at least 50 weight-% isopentane, based on the total weight of said hydrocarbon feed stream.

23. A process in accordance with claim 12 wherein said hydrocarbon feed stream comprises in the range of from about 50 to about 95 weight-% isopentane, based on the total weight of said hydrocarbon feed stream.

24. A process in accordance with claim 12 wherein said hydrocarbon feed stream comprises in the range of from about 80 to about 98.5 weight-% isopentane, based on the total weight of said hydrocarbon feed stream.

25. A process in accordance with claim 12 wherein said conversion conditions include a temperature in the range of from about 100° F. to about 1000° F.

26. A process in accordance with claim 12 wherein said conversion conditions include a temperature in the range of from about 140° F. to about 250° F.

27. A process in accordance with claim 12 wherein said conversion conditions include a temperature in the range of from about 150° F. to about 220° F.

28. A process in accordance with claim 12 wherein said C₄ paraffin of said product stream is isobutane and said C₆ paraffin of said product stream is a hexane isomer.

29. A process in accordance with claim 12 wherein said initiator is selected from the group consisting of: 1) an olefin having in the range of from 2 to 20 carbon atoms per molecule, 2) an alkyl halide wherein said alkyl halide has in the range of from 2 to 20 carbon atoms per molecule, and combinations thereof.
